(19) United States
(12) Patent Application Publication
(10) Pub. No.: US 2023/0226017 A1
(43) Pub. Date: Jul. 20, 2023

(54) METHODS OF TREATING A CORONAVIRUS INFECTION

(71) Applicant: The General Hospital Corporation, Boston, MA (US)

(72) Inventor: David R. Elmaleh, Newton, MA (US)

(21) Appl. No.: 18/007,822

(22) PCT Filed: Jun. 4, 2021
(86) PCT No.: PCT/US21/35936

§ 371 Date: Dec. 2, 2022

Related U.S. Application Data
(60) Provisional application No. 63/034,637, filed on Jun. 4, 2020.

(51) Int. Cl. A61K 31/353; A61K 31/664; A61K 31/52; A61K 31/506; A61K 9/00; A61P 31/14

(52) U.S. Cl. CPC A61K 31/353; A61K 31/664; A61K 31/52; A61K 31/506; A61K 9/0019; A61K 9/0075; A61P 31/14

(57) ABSTRACT

The present disclosure relates to a pharmaceutical composition, such as a dry powder inhalation formulation or an injectable formulation, comprising a mixture of an antiviral agent and a mast cell stabilizer. The present disclosure relates to a codrug comprising a residue of an antiviral agent covalently bonded via a labile bond to a residue of a compound of Formula (I) or Formula (II). The present disclosure further relates to a method of administering an antiviral agent and a Formula I/II compound, a pharmaceutical composition, or a codrug to treat coronavirus infection and/or associated inflammation.

### CROSS-REFERENCE TO RELATED APPLICATIONS

This application claims priority to U.S. Provisional Patent Application No. 63/034,637, filed Jun. 4, 2020, the entire contents of which are incorporated herein by reference.

### BACKGROUND

Severe cases of novel coronavirus disease 2019 (COVID-19) are characterized by an overactive inflammatory response to the severe acute respiratory syndrome coronavirus 2 (SARS-CoV-2) pathogen. As the collective human immune system has little to no pre-existing immunity to SARS-CoV-2, the immune response to release inflammatory cytokines is exaggerated and overactive (i.e., the cytokine storm) and is correlated to the severity of COVID-19, culminating in breathing difficulty, pneumonia, acute respiratory distress syndrome (ARDS), viral sepsis, and potentially death. As there is no known effective treatment yet available to stop or reverse the worst symptomatic cases of COVID-19, there exists a need for a therapy to simultaneously dampen the immunoinflammatory response of the cytokine storm and curb in vivo replication of SARS-CoV-2 or coronaviruses in general, such as SARS-CoV, MERS-CoV, possible future strains of SARS-CoV-2 and other viral infection associated with massive immunogenic cytokine release.

### SUMMARY OF THE INVENTION

In one aspect, the present disclosure relates to a pharmaceutical composition comprising an antiviral agent and a mast cell stabilizer (e.g., a compound of Formula I or Formula II:

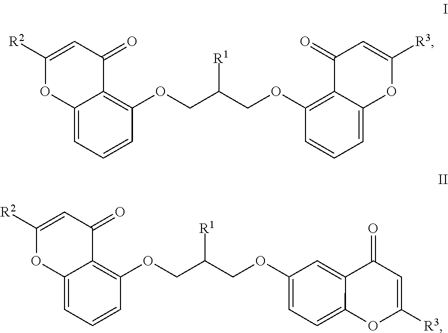

wherein R¹, R² and R³ are defined herein or a pharmaceutically acceptable salt thereof).

In another aspect, the present disclosure relates to a codrug comprising a residue of an antiviral agent covalently bonded via a labile bond to a residue of a mast cell stabilizer (e.g., a compound of Formula I or Formula II). A pharmaceutical composition comprising a codrug disclosed herein and a pharmaceutically acceptable excipient is also provided.

In yet another aspect, the present disclosure relates to a method of treating a coronavirus infection and/or associated inflammation, comprising administering to a subject in need thereof a codrug or a pharmaceutical composition disclosed herein.

In yet another aspect, the present disclosure relates to a method of treating a coronavirus infection and/or associated inflammation, comprising conjointly administering to a subject in need thereof an antiviral agent and a compound of Formula I or Formula II or a pharmaceutically acceptable salt thereof.

### DETAILED DESCRIPTION OF THE INVENTION

### Overview

The severe acute respiratory syndrome coronavirus 2 (SARS-CoV-2) responsible for coronavirus disease 2019 (COVID-19) is a positive sense single-stranded RNA virus.

For patients exhibiting severe illness, there are high levels of pro-inflammatory cytokines in blood plasma, correlating an overactive immune response of cytokine release (cytokine storm) to the severity of COVID-19 disease. The inflammation caused by the cytokine storm within lung epithelial tissue can lead to the deadliest aspects of COVID-19, notably activated lymphocyte infiltration of the lungs and heart resulting in acute respiratory distress syndrome (ARDS) and cardiac failure. Cromolyn is FDA-approved in many formulations as a mast cell stabilizer for the management of asthma. Cromolyn's mechanism of action is to block mast cell degranulation and release of histamine, suppressing lymphocyte activation and release of cytokines in an immune response. WO 2020/051322 discloses treatment of the cytokine release syndrome by several compound types, including mast cell stabilizers. WO 2020/051322 is hereby incorporated by reference in its entirety and particular with respect to the mast cell stabilizers disclosed therein.

In one aspect, disclosed herein is a method of treating a coronavirus infection and/or associated inflammation, comprising administering to a subject in need thereof an antiviral agent and a mast cell stabilizer (e.g., a compound of Formula I or Formula II, cromolyn). In certain aspects, also described herein is a pharmaceutical composition comprising an antiviral agent, e.g., remdesivir, and an anti-HIV drug, and a mast cell stabilizer (e.g., a compound of Formula I or Formula II, cromolyn). Such compositions and methods seek to limit the viral load of COVID-19 patients via an antiviral agent and to inhibit the cytokine storm associated with severity of disease via a mast cell stabilizer (e.g., a compound of Formula I or Formula II, cromolyn). In a further aspect, the instant disclosure also relates to a codrug comprising a residue of an antiviral agent and a residue of a mast cell stabilizer (e.g., a compound of Formula I or Formula II, cromolyn).

### I. Exemplary Mast Cell Stabilizers and Antiviral Agents

In some embodiments, disclosed herein is a compound of Formula I or Formula II.

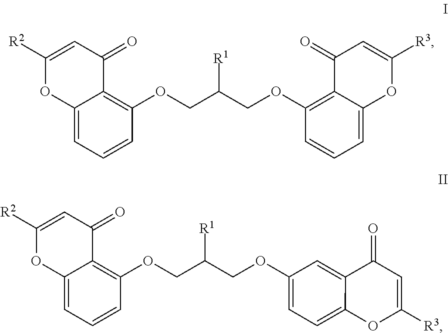

wherein
R¹ is halogen, OH, or —OC(O)C₁₋₅alkyl
R² and R³ are each independently selected from CO₂R⁴ and CH₂OR⁵;
R⁴ is Li, Na, K, H, C₁₋₅alkyl, or —CH₂CO(C₁₋₅alkyl); and
R⁵ is H or —C(O)(C₁₋₅alkyl),
or a pharmaceutically acceptable salt thereof.

In some embodiments, R¹ is halogen, for example, R¹ is F. In certain preferred embodiments, R¹ is OH. In some embodiments, R¹ is —OC(O)C₁₋₄alkyl, such as —OC(O)Me.

In certain embodiments, R² and R³ is each independently —CO₂R⁴. In some embodiments, R⁴ is Li, Na, K, or NH₄, for example, R⁴ is Na. In certain embodiments, R⁴ is H. In some embodiments, R⁴ is C₁₋₅alkyl. In certain embodiments, R⁴ is —CH₂CO(C₁₋₅alkyl);

In certain embodiments, R² and R³ is each independently —CH₂OR⁵. In some embodiments, R⁵ is H. In certain embodiments, R⁵ is —C(O)(C₁₋₅alkyl).

In some embodiments, C₁₋₅alkyl is methyl, ethyl, or t-butyl.

In certain embodiments, the compound of Formula I is selected from:

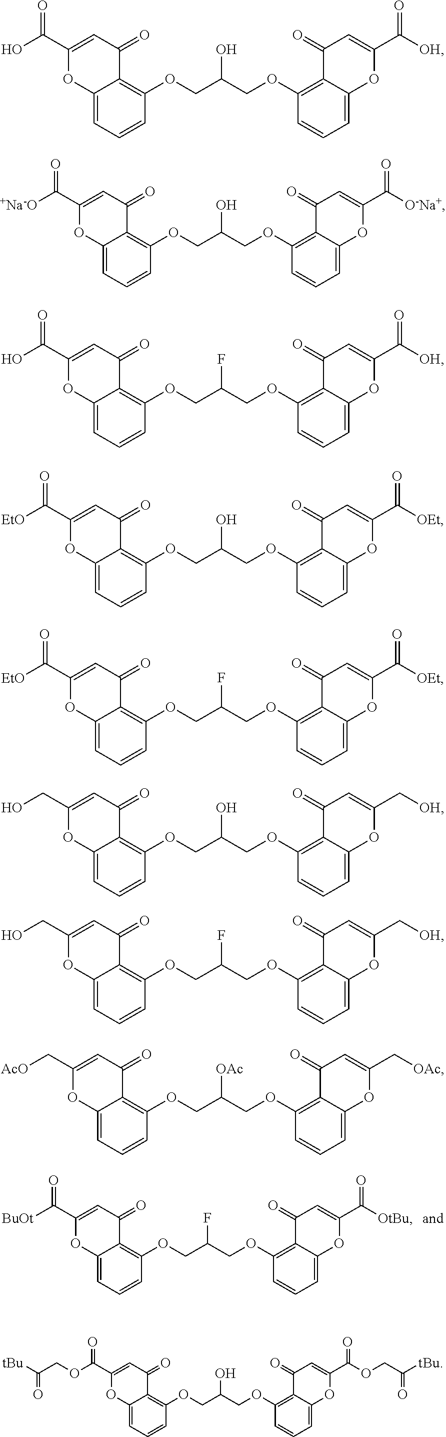

In certain embodiments, the compound of Formula I is

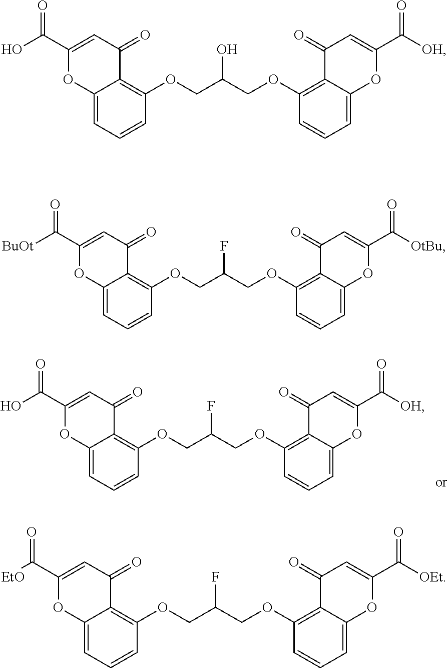

In certain embodiments, the compound of Formula I is

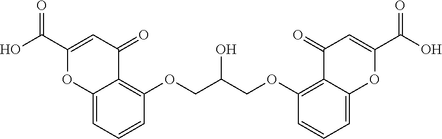 [SMILES: O=C(O)c1cc(=O)c2c(OCC(O)COc3cccc4oc(C(=O)O)cc(=O)c34)cccc2o1]

or a pharmaceutically acceptable salt thereof, also known as cromolyn. In certain embodiments, the compound of Formula I is cromolyn or a fluorinated compound thereof or a pharmaceutically acceptable salt thereof.

In some embodiments, the compound of Formula I is

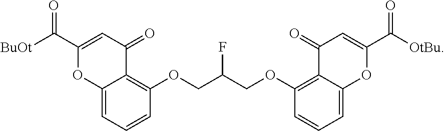

In some embodiments, the compound of Formula I is

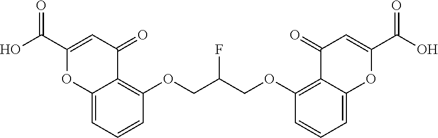 [SMILES: O=C(O)c1cc(=O)c2c(OCC(F)COc3cccc4oc(C(=O)O)cc(=O)c34)cccc2o1]

or a pharmaceutically acceptable salt thereof.

In some embodiments, the compound of Formula I is a Li, Na, or K salt or a C₁₋₅alkyl ester of cromolyn.

In certain embodiments, the compound of Formula I is

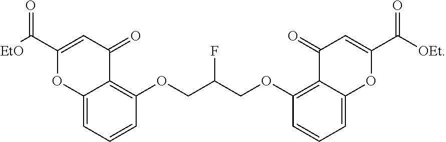 [SMILES: CCOC(=O)c1cc(=O)c2c(OCC(F)COc3cccc4oc(C(=O)OCC)cc(=O)c34)cccc2o1]

In some embodiments, the antiviral agent is selected from remdesivir (RDV), abacavir, atazanavir, bictegravir (BIC), cobicistat (GS-39250), darunavir (DRV), didanosine (ddl), dolutegravir (DTG), doravirine (MK-1439), efavirenz (EFV), elvitegravir (EVG), emtricitabine (FTC), enfuvirtide (INN), fosamprenavir, inidinavir (IDV), lamivudine (3TC), lopinavir, maraviroc, nelfinavir (NFV), Nevirapine (NVP), raltegravir (RAL), rilpivirine (TMC278), ritonavir, saquinavir (SQV), tenofovir alafenarmide (TAF), tefofovir disoproxil fumerate (TDF), tipranvir (TPV), and zidovudine (ZDV). In certain preferred embodiments,

the antiviral agent is remdesivir:

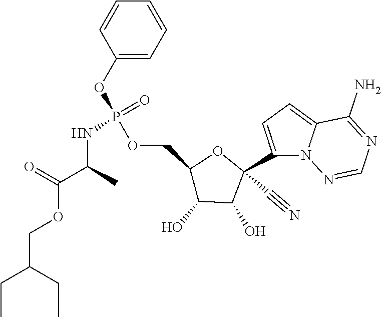 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof.

In certain embodiments, the antiviral agents are anti-HIV drugs. Exemplary anti-HIV drugs are provided in Table 1.

TABLE 1

| | | | Exemplary |
|---|---|---|---|
| | | Mechanism | Modes of |
| Name | Structure | of Action | Delivery |
| Abacavir, or ABC, (Ziagen) | 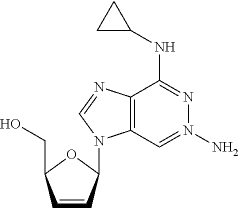 | Nucleoside analog Reverse Transcriptase Inhibitor (NRTI) | By mouth, solution or tablets |
| Didanosine, or ddl, (Videx) | 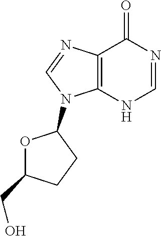 [SMILES: O=c1nc[nH]c2c1ncn2[C@H]1CC[C@@H](CO)O1] | NRTI | By mouth capsule |
| Emtricitabine, or FTC, (Emtriva) | 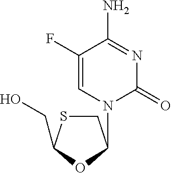 [SMILES: Nc1nc(=O)n([C@@H]2CS[C@H](CO)O2)cc1F] | NRTI | By mouth capsule |
| Lamivudine, or 3TC, (Epivir) | 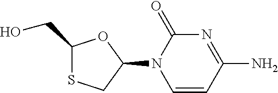 [SMILES: Nc1ccn([C@H]2CS[C@@H](CO)O2)c(=O)n1] | NRTI | By mouth, solution or tablet |
| Stavudine, or d4T, (Zerit) | 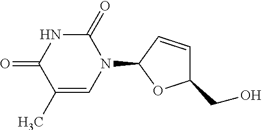 [SMILES: Cc1cn([C@H]2C=C[C@@H](CO)O2)c(=O)[nH]c1=O] | NRTI | By mouth capsule |
| Tenofovir alafenamide, or TAF, (Vemlidy) | 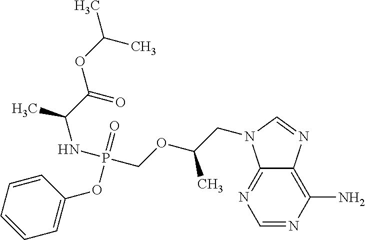 [SMILES: CC(C)OC(=O)[C@H](C)NP(=O)(CO[C@H](C)Cn1cnc2c(N)ncnc21)Oc1ccccc1] | NRTI | By mouth tablet |
| Tenofovir disoproxil fumerate, or TDF, (Viread) | 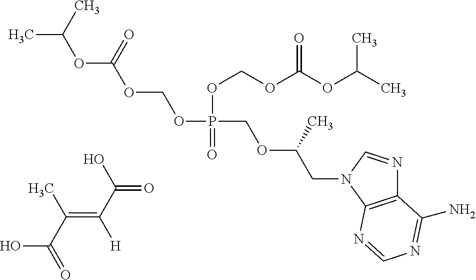 | NRTI | By mouth tablet |
| Zidovudine, or ZDV, (Retrovir) | 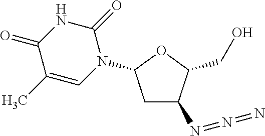 | NRTI | By mouth, slow intravenous infusion, rectal suppository |
| Atazanavir, (Reyataz) | 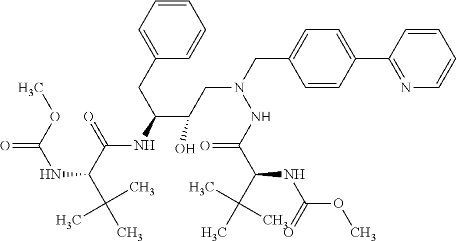 [SMILES: COC(=O)N[C@H](C(=O)N[C@@H](Cc1ccccc1)[C@@H](O)CN(Cc1ccc(-c2ccccn2)cc1)NC(=O)[C@@H](NC(=O)OC)C(C)(C)C)C(C)(C)C] | HIV Protease Inhibitor | By mouth capsule |
| Darunavir, or DRV, (Prezista) | 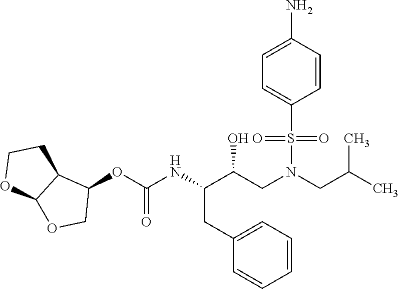 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1] | HIV Protease Inhibitor | By mouth capsule |
| Fosamprenavir - a pro-drug of amprenavir, (Lexiva) | 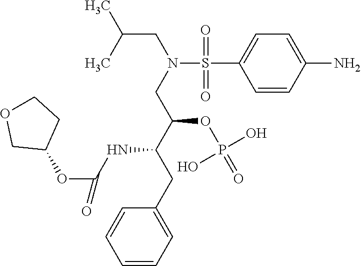 [SMILES: CC(C)CN(C[C@@H](OP(=O)(O)O)[C@H](Cc1ccccc1)NC(=O)O[C@H]1CCOC1)S(=O)(=O)c1ccc(N)cc1] | HIV Protease Inhibitor | By mouth capsule |
| Lopinavir - fixed dose combination with ritonavir, (Kaletra) | 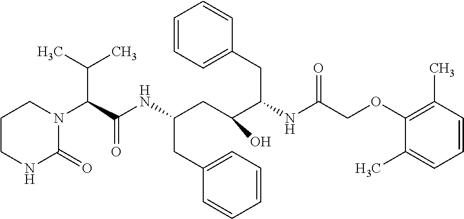 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O] | HIV Protease Inhibitor | By mouth capsule |
| Ritonavir, or RTV, (Norvir) | 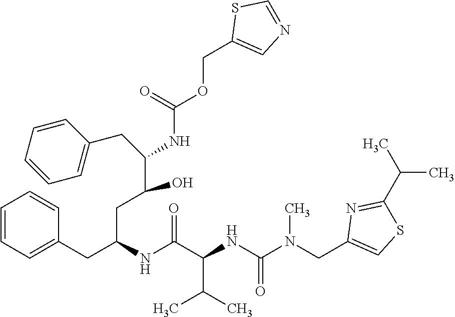 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1] | HIV Protease Inhibitor | By mouth capsule |
| Tipranavir, or TPV, (Aptivus) | 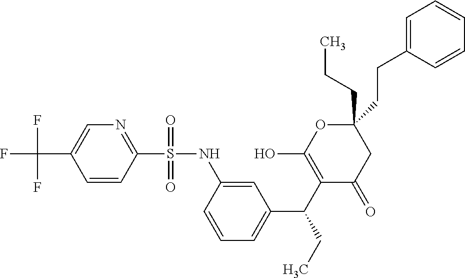 [SMILES: CCC[C@@]1(CCc2ccccc2)CC(=O)C([C@H](CC)c2cccc(NS(=O)(=O)c3ccc(C(F)(F)F)cn3)c2)=C(O)O1] | HIV Protease Inhibitor | By mouth capsule |
| Bictegravir, or BIC, (Biktarvy - combination drug of bictegravir/ emtricitabine/tenofovir alafenamide) | 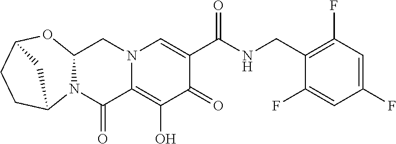 [SMILES: O=C(NCc1c(F)cc(F)cc1F)c1cn2c(c(O)c1=O)C(=O)N1[C@H]3CC[C@H](C3)O[C@@H]1C2] | HIV Integrase Strand Transfer Inhibitor | By mouth tablet (50 mg bictegravir, 200 mg emtricitabine, 25 mg tenofovir alafenamide) |
| Dolutegravir, or DTG, (Tivicay) | 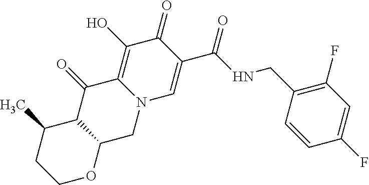 | HIV Integrase Strand Transfer Inhibitor | By mouth tablet |
| Elvitegravir, or EVG, (Vitekta) | 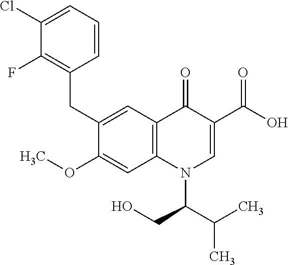 [SMILES: COc1cc2c(cc1Cc1cccc(Cl)c1F)c(=O)c(C(=O)O)cn2[C@H](CO)C(C)C] | HIV Integrase Strand Transfer Inhibitor | By mouth tablet |
| Raltegravir, or RAL, (Isentress) | 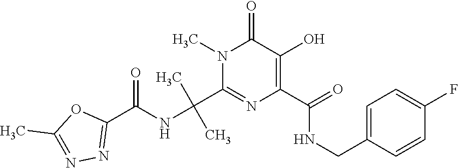 [SMILES: Cc1nnc(C(=O)NC(C)(C)c2nc(C(=O)NCc3ccc(F)cc3)c(O)c(=O)n2C)o1] | HIV Integrase Strand Transfer Inhibitor | By mouth tablet |
| Doravirine, or MK-1439, (Pifeltro) | 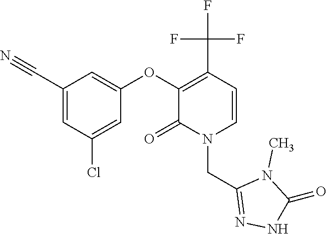 [SMILES: Cn1c(Cn2ccc(C(F)(F)F)c(Oc3cc(Cl)cc(C#N)c3)c2=O)n[nH]c1=O] | Non- Nucleoside Reverse Transcriptase Inhibitor | By mouth tablet |
| Efavirenz, or EFV, (Sustiva) | 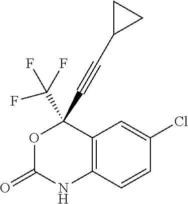 [SMILES: O=C1Nc2ccc(Cl)cc2[C@@](C#CC2CC2)(C(F)(F)F)O1] | Non- Nucleoside Reverse Transcriptase Inhibitor | By mouth capsule, tablet |
| Nevirapine, or NVP, (Viramune) | 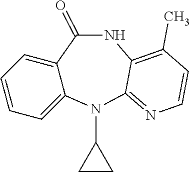 [SMILES: Cc1ccnc2c1NC(=O)c1ccccc1N2C1CC1] | Non- Nucleoside Reverse Transcriptase Inhibitor | By mouth tablet |
| Rilpivirine, or TMC278, (Edurant) | 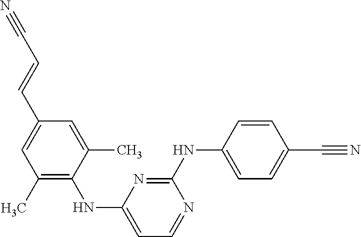 [SMILES: Cc1cc(/C=C/C#N)cc(C)c1Nc1ccnc(Nc2ccc(C#N)cc2)n1] | Non- nucleoside Reverse Transcriptase Inhibitor | By mouth tablet |
| Cobicistat, or GS-9350, (Tybost) | 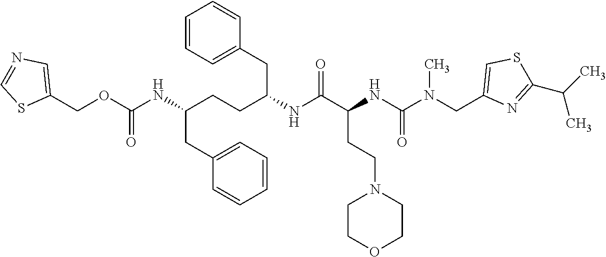 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1] | Cytochrome P450 3A (CYP3A) inhibitor - used to increase blood plasma levels of co- administered HIV antivirals | By mouth tablet, in combined formulations with other HIV antiviral medications |
| Inidinavir, or IDV, (Crixivan) | 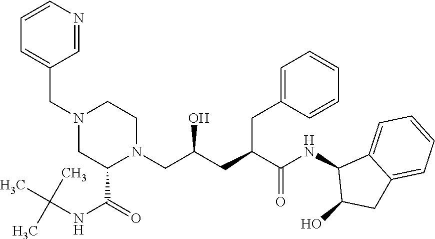 [SMILES: CC(C)(C)NC(=O)[C@@H]1CN(Cc2cccnc2)CCN1C[C@@H](O)C[C@@H](Cc1ccccc1)C(=O)N[C@H]1c2ccccc2C[C@H]1O] | HIV Protease Inhibitor | By mouth capsule |
| Nelfinavir, or NFV, (Viracept) | 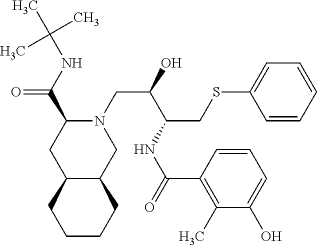 [SMILES: Cc1c(O)cccc1C(=O)N[C@@H](CSc1ccccc1)[C@H](O)CN1C[C@H]2CCCC[C@H]2C[C@H]1C(=O)NC(C)(C)C] | HIV Protease Inhibitor | By mouth tablet |
| Saquinavir, or SQV, (Invirase, Fortovase) | 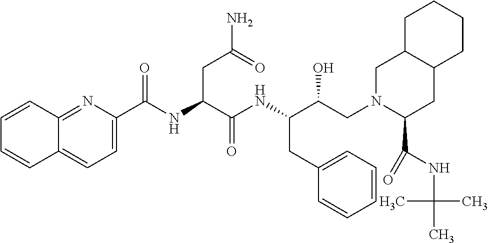 [SMILES: CC(C)(C)NC(=O)[C@@H]1CC2CCCCC2CN1C[C@@H](O)[C@H](Cc1ccccc1)NC(=O)[C@H](CC(N)=O)NC(=O)c1ccc2ccccc2n1] | HIV Protease Inhibitor HIV Fusion Inhibitor | By mouth capsule, tablet |
| Enfuvirtide, or INN, (Fuzeon) | 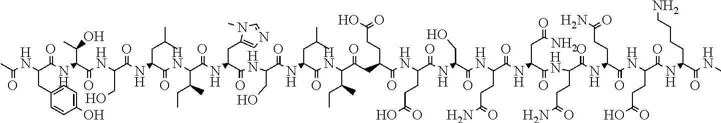 [SMILES: CC[C@H](C)C(NC(=O)[C@H](CC(C)C)NC(=O)C(CO)NC(=O)[C@H](Cc1cncn1C)NC(=O)C(NC(=O)[C@H](CC(C)C)NC(=O)C(CO)NC(=O)[C@@H](NC(=O)C(Cc1ccc(O)cc1)NC(C)=O)[C@@H](C)O)[C@@H](C)CC)C(=O)C[C@@H](CCC(=O)O)C(=O)NC(CCC(=O)O)C(=O)N[C@@H](CO)C(=O)NC(CCC(N)=O)C(=O)N[C@@H](CC(N)=O)C(=O)NC(CCC(N)=O)C(=O)N[C@@H](CCC(N)=O)C(=O)NC(CCC(=O)O)C(=O)N[C@@H](CCCCN)C(=O)NC] | | Subcutaneous injection |
| | 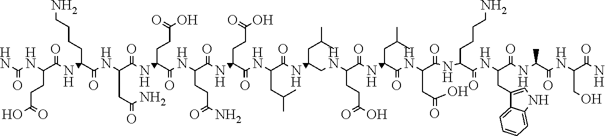 | | |
| | 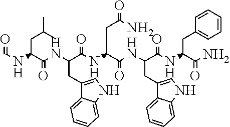 [SMILES: CC(C)C[C@H](NC=O)C(=O)NC(Cc1c[nH]c2ccccc12)C(=O)N[C@@H](CC(N)=O)C(=O)NC(Cc1c[nH]c2ccccc12)C(=O)N[C@@H](Cc1ccccc1)C(N)=O] | | |
| Maraviroc, (Selzentry, Celsentri) | 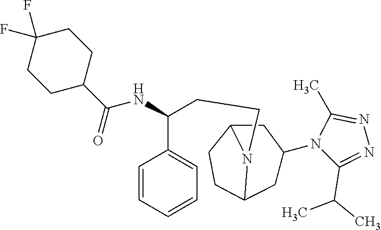 [SMILES: Cc1nnc(C(C)C)n1C1CC2CCC(C1)N2CC[C@H](NC(=O)C1CCC(F)(F)CC1)c1ccccc1] | HIV Fusion Inhibitor | By mouth tablets or oral solution |

In some embodiments, the antiviral drug is not remdesivir (RDV), tilorone, favipiravir, IFNα, IFNβ, IFNγ, peginterferon-α, peginterferon-β, ribavirin, lopinavir/ritonavir, camostat mesylate, TAK888, abacavir, acyclovir, adefovir, amantadine, rintatolimod (Ampligen), amprenavir, umifenovir (Arbidol), or atazanavir.

### II. Codrugs

In some embodiments, disclosed herein is a codrug comprising a residue of an antiviral agent covalently bonded via a labile bond to a residue of a compound of Formula I or Formula II.

In certain embodiments, the residue of the antiviral agent is covalently bonded via an amide bond to a residue of a compound of Formula I or Formula II. In certain such embodiments, the amide bond is formed between a functional group of the antiviral compound and a functional group at R² or R³ of the compound of Formula I or Formula II. Not wishing to be bound by theory, it is expected that the hydrolysis of the labile bond (e.g., amide bond) will form in situ the corresponding antiviral agent and the a compound of Formula I or Formula II after administration to a subject.

In certain embodiments, the codrug is selected from remdesivir-cromolyn, abacavir-cromolyn, emtricitabine-cromolyn, lamivudine-cromolyn, tenofovir alafenamide-cromolyn, tenofovir disoproxil-cromolyn fumarate, darunavir-cromolyn, and fosamprenavir-cromolyn. In certain preferred embodiments, the codrug is remdesivir-cromolyn.

In certain embodiments, the codrug is selected from

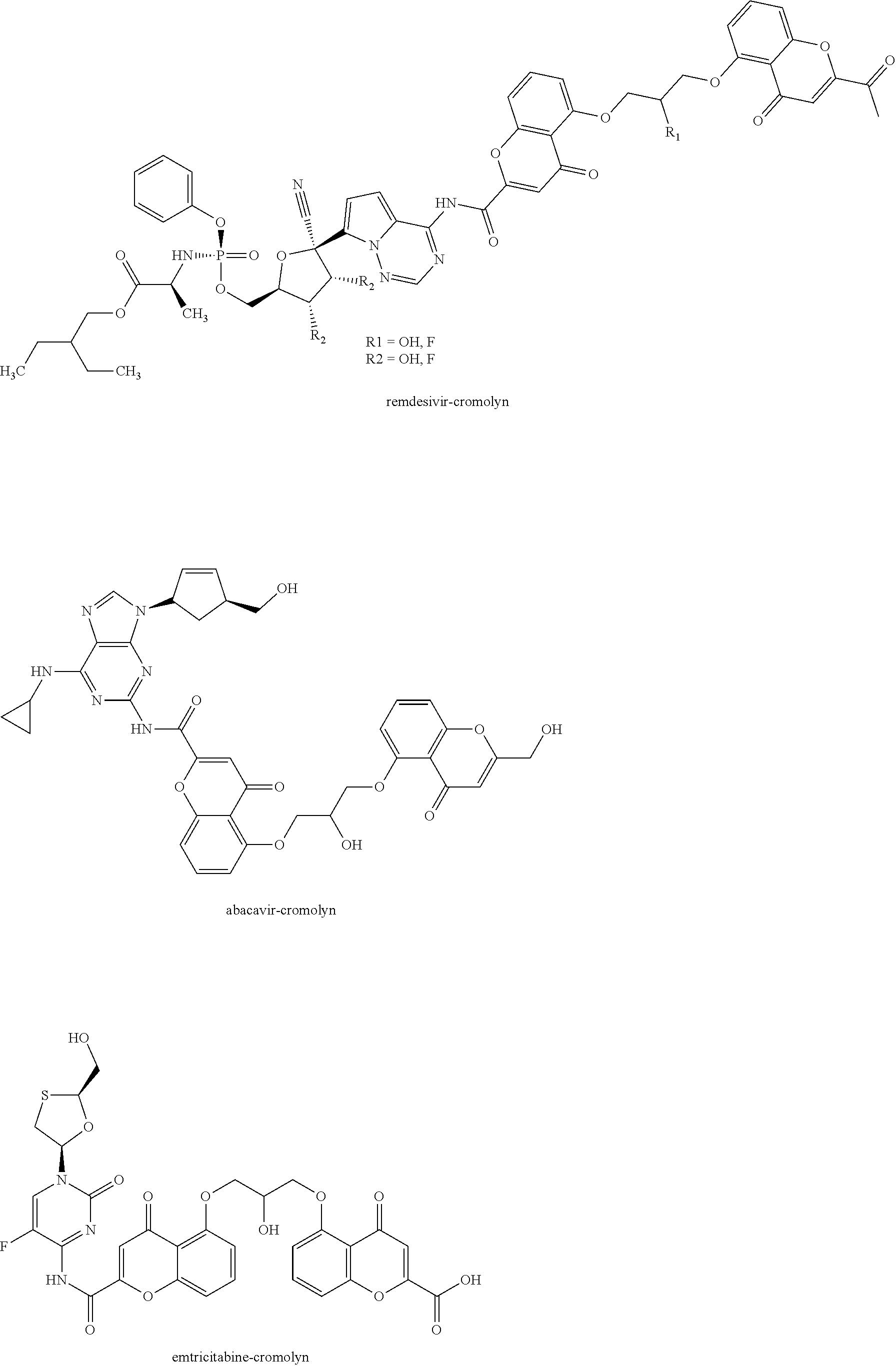

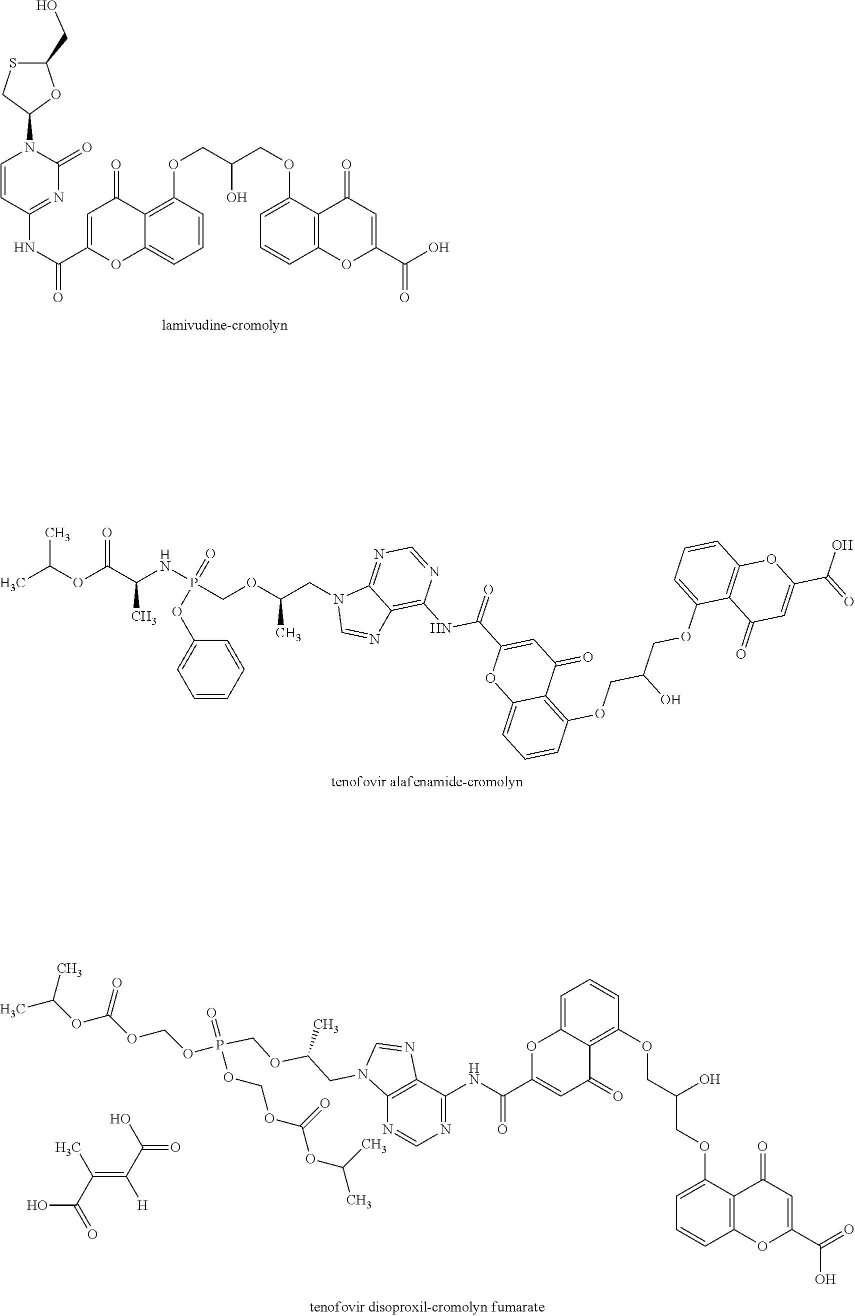

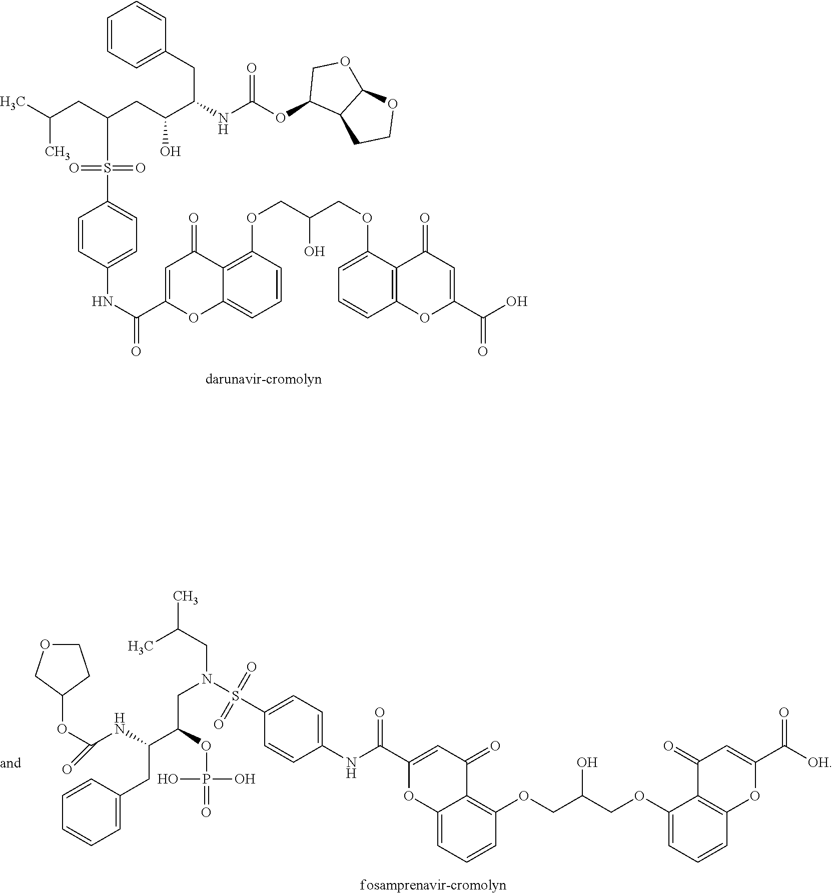 [SMILES: CC(C)CC(C[C@@H](O)[C@H](Cc1ccccc1)NC(=O)O[C@H]1CO[C@H]2OCC[C@H]21)S(=O)(=O)c1ccc(NC(=O)c2cc(=O)c3c(OCC(O)COc4cccc5oc(C(=O)O)cc(=O)c45)cccc3o2)cc1 ; CC(C)CN(C[C@@H](OP(=O)(O)O)[C@H](Cc1ccccc1)NC(=O)OC1CCOC1)S(=O)(=O)c1ccc(NC(=O)c2cc(=O)c3c(OCC(O)COc4cccc5oc(C(=O)O)cc(=O)c45)cccc3o2)cc1]

In certain embodiments, the co-drug is remdesivir-cromolyn, or fluorinated codrug thereof, or a pharmaceutically acceptable salt thereof

### III. Pharmaceutical Compositions

In certain embodiments, the present disclosure provides a pharmaceutical composition, comprising an antiviral agent and compound of Formula I or Formula II or a pharmaceutically acceptable salt thereof. The present disclosure also provides a pharmaceutical composition comprising a codrug disclosed herein and a pharmaceutically acceptable excipient.

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, the contents of which are incorporated herein by reference in their entirety, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference in their entirety. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, a combined pharmaceutical composition comprises an antiviral agent and a mast cell stabilizer. The mass ratio between the antiviral agent and the mast cell stabilizer in the pharmaceutical composition is about 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:10, 1:9, 1:8, 1:7, 1:6, 1:5, 1:4, 1:3, 1:2, and 1:1. The mass ratio between the antiviral agent and the mast cell stabilizer in the pharmaceutical composition is also about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, and 1:1.

In some embodiments, a combined pharmaceutical composition comprises an antiviral agent and a compound of Formula I or Formula II and the mass ratio between the antiviral agent and the compound of Formula I or Formula II in the pharmaceutical composition is from about 10:1 to about 1:10. In some embodiments, the mass ratio between the antiviral agent and the compound of Formula I or Formula II in the pharmaceutical composition is about 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:10, 1:9, 1:8, 1:7, 1:6, 1:5, 1:4, 1:3, 1:2, and 1:1. The mass ratio between the antiviral agent and the compound of Formula I or Formula II in the pharmaceutical composition is also about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, and 1:1.

In some embodiments, the pharmaceutical composition comprises about 10 mg to 100 mg, about 10 mg to 80 mg, about 10 mg to 60 mg, about 10 mg to 40 mg, or about 20 mg to 40 mg of the compound of Formula I or Formula II (e.g., cromolyn). In certain embodiments, the pharmaceutical composition comprises about 10 mg, about 15 mg, about 20 mg, about 25 mg, about 30 mg, about 35 mg, about 40 mg, about 45 mg, or about 50 mg of the compound of Formula I or Formula II (e.g., cromolyn). In preferred embodiments, the pharmaceutical composition comprises about 30 mg of the compound of Formula I or Formula II (e.g., cromolyn).

In some embodiments, the pharmaceutical composition comprises about 10 mg to 100 mg, about 10 mg to 80 mg, about 10 mg to 60 mg, about 20 mg to 80 mg, or about 30 mg to 60 mg of the antiviral agent. In certain embodiments, the pharmaceutical composition comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg of the antiviral agent. In preferred embodiments, the pharmaceutical composition comprises about 50 mg of the antiviral agent.

In some embodiments, the pharmaceutical composition is an inhalation composition. In some embodiments, the pharmaceutical composition is a dry powder inhalation composition. In some embodiments, the pharmaceutical composition is an injectable formulation. In some embodiments, the pharmaceutical composition is an intravenous infusion formulation. In some embodiments, the pharmaceutical composition is a subcutaneously injectable formulation.

### Inhaler

In certain aspects, provided herein is an inhaler device comprising a pharmaceutical composition disclosed herein. In accordance with one embodiment of the present disclosure, the pharmaceutical composition is in the form of a dry powder. Preferably, the dry powder is dispensed by a dry powder inhaler (DPI). In a further embodiment, the inhaler is an active inhaler. In yet another embodiment, the inhaler is a breath actuated inhaler device.

### IV. Methods of Use

In certain aspects, provided herein are methods of treating a coronavirus infection and/or associated inflammation comprising administering to a subject in need thereof an antiviral agent and a compound of Formula I or Formula II.

SARS-CoV replication process may be interrupted via remdesivir intravenous treatment as seen with SARS-CoV and MERS-CoV. Emerging reports and clinical trials of COVID-19 patients treated with remdesivir have had initial success with shortening the symptomatic stage of the disease, presumably due to an antiviral effect. Although remdesivir shows promise to limit the replication of SARS-CoV-2 in COVID-19 patients, it is not yet FDA-approved for COVID-19 or any other indication. Therefore, it is important to consider other antivirals for treating a coronavirus infection, in addition to remdesivir, that may also be able to limit SARS-CoV-2 proliferation in subjects.

In certain embodiments, the antiviral agent and the compound of Formula I or Formula II are administered conjointly. In certain embodiments, conjoint administration of the antiviral agent and the compound of Formula I or Formula II provides improved efficacy relative to each individual administration of the compound Formula I or II or the antiviral agent. In certain such embodiments, the conjoint administration provides an additive effect, wherein an additive effect refers to the sum of each of the effects of individual administration of the compound of the invention and the one or more additional therapeutic agent(s). In other embodiments, the conjoint administration of a compound of the invention reduces or ameliorates the side effects of the additional therapeutic agent.

The antiviral agent may be administered simultaneously with the compound of Formula I or Formula II. Alternatively, the antiviral agent may be administered prior to administration the compound of Formula I or Formula II. Alternatively, still, the antiviral agent may be administered following the administration of the compound of Formula I or Formula II.

In certain other aspects, provided herein are methods of treating a coronavirus infection and/or associated inflammation, comprising administering pharmaceutical composition disclosed herein. In yet another aspect, provided herein are methods of treating a coronavirus infection and/or associated inflammation, comprising administering a codrug disclosed herein.

In certain embodiments, the compound, antiviral agent, codrug, or pharmaceutical composition is administered intravenously, intrathecally, subcutaneously, intramuscularly, intranasally, or orally.

In some embodiments, the pharmaceutical composition is administered for a treatment period from 1 day to 42 days. In some embodiments, the pharmaceutical composition is administered for a treatment period from 21 days to 35 days. In some embodiments, the pharmaceutical composition is administered for a treatment period from 3 days to 10 days. In some embodiments, the pharmaceutical composition is administered for a treatment period for about 7 days. In some embodiments, the pharmaceutical composition is administered for a treatment period of about 28 days.

The effective amount of a compound of the invention in such a therapeutic method is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day. In some embodiments, the effective amount of a compound of the invention in such a therapeutic method is about 2 mg/kg/day, about 5 mg/kg/day, about 7.5 mg/kg/day, about 10 mg/kg/day, about 12.5 mg/kg/day, about 15 mg/kg/day, or about 20 mg/kg/day.

In some embodiments, the effective amount of the compound of Formula I or Formula II (e.g., cromolyn) is about 10 mg to 100 mg, about 10 mg to 80 mg, about 10 mg to 60 mg, about 10 mg to 40 mg, or about 20 mg to 40 mg. In certain embodiments, the effective amount of the compound of Formula I or Formula II (e.g., cromolyn) is about 10 mg, about 15 mg, about 20 mg, about 25 mg, about 30 mg, about 35 mg, about 40 mg, about 45 mg, or about 50 mg of the compound of Formula I or Formula II (e.g., cromolyn). In preferred embodiments, the effective amount of the compound of Formula I or Formula II (e.g., cromolyn) is about 30 mg.

In some embodiments, the effective amount of the antiviral agent is about 10 mg to 100 mg, about 10 mg to 80 mg, about 10 mg to 60 mg, about 20 mg to 80 mg, or about 30 mg to 60 mg of the antiviral agent. In certain embodiments, the effective amount of the antiviral agent is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg of the antiviral agent. In preferred embodiments, the effective amount of the antiviral agent is about 50 mg of the antiviral agent.

In certain embodiments, the mass ratio between the antiviral agent and the compound of Formula I or Formula II is about 5:3. In certain embodiments, the antiviral agent and the compound of Formula I or Formula II can be administered by intravenous infusion or by intravenous injection and the mass ratio between the antiviral agent and the compound of Formula I or Formula II is about 2:1.

In some embodiments, the pharmaceutical composition is administered from 1 to 5 times a day. In some embodiments, the pharmaceutical composition is administered 3 times a day. In some embodiments, the pharmaceutical composition is administered with a frequency from about every 2 hours to about every 6 hours. In some embodiments, the pharmaceutical composition is administered with a frequency of about every 4 hours.

In some embodiments, the method comprises administering the pharmaceutical composition by intravenous infusion, by intravenous injection, by subcutaneous injection, by intramuscular injection, by intraperitoneal injection, orally, sublingually, buccally, or by inhalation. In some embodiments, the method comprises administering the pharmaceutical composition by intravenous infusion or by intravenous injection. In some embodiments, the method comprises administering the pharmaceutical composition orally. In some preferred embodiments, the method comprises administering the pharmaceutical composition by inhalation. In some embodiments, the method comprises administering the pharmaceutical by inhalation using a nebulizer.

In some embodiments, the coronavirus is selected from SARS-CoV, MERS-CoV, HCoV, HKU1, and SARS-CoV-2. In some embodiments, the coronavirus is selected from SARS-CoV, MERS-CoV, and SARS-CoV-2. In preferred embodiments, the coronavirus is SARS-CoV-2.

In some embodiments, the inflammation is selected from acute respiratory distress syndrome (ARDS), pneumonia, myocarditis, haemophagocytic lymphohistiocytosis (sHLH), kidney failure, septic shock, and sepsis. In some embodiments, the associated inflammation is pneumonia. In some embodiments, the associated inflammation condition is ARDS. In some embodiments, the associated inflammation is myocarditis. In some embodiments, at least one inflammation is sepsis.

In some embodiments, the subject is aged 18-75 years, inclusive.

In some embodiments, the subject has SARS-CoV-2 infection, which has been confirmed by reverse-transcription polymerase chain reaction (RT-PCR) from respiratory tract or blood specimens.

Methods of Preparation.

Compounds of the invention may be prepared according to the synthetic procedures described below. In cases where the synthetic intermediates and final products of Formula I described below contain potentially reactive functional groups, for example amino, hydroxy, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). Such protecting group manipulations are assumed in the discussion below and not usually described explicitly. Generally, reagents in the reaction schemes are used in equimolar amounts; however, in certain cases it may be desirable to use an excess of one reagent to drive a reaction to completion. This is especially the case when the excess reagent can be readily removed by evaporation or extraction. Bases employed to neutralize HCl in reaction mixtures are generally used in slight to substantial excess (1.05-5 equivalents).

Compounds of the invention can be prepared employing conventional methods that utilize readily available reagents and starting materials. The reagents used in the preparation of the compounds of this invention can be either commercially obtained or can be prepared by standard procedures described in the literature. The compounds of the invention may be made according to the general and exemplary schemes provided herein.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

When any variable (e.g., aryl, heterocyclyl, R², R, etc.) occurs more than once in a compound, its definition on each occurrence is independent of any other occurrence.

Unless specifically stated or obvious from context, as used herein, the term “or” is understood to be inclusive. Unless specifically stated or obvious from context, as used herein, the terms “a”, “an”, and “the” are understood to be singular or plural.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as alkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc.

The terms “halo” and “halogen” as used herein means halogen and includes chloro, fluoro, bromo, and iodo.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. It will be understood by those skilled in the art that substituents can themselves be substituted, if appropriate. Unless specifically stated as “unsubstituted,” references to chemical moieties herein are understood to include substituted variants. For example, reference to an “alkyl” group or moiety implicitly includes both substituted and unsubstituted variants.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

As used herein, the phrase “conjoint administration” refers to any form of administration of two or more different therapeutic compounds such that the second compound is administered while the previously administered therapeutic compound is still effective in the body or while the side effects of the previously administered therapeutic compound are still evident in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

As used herein, the term “coronavirus” refers to a virus belonging to the subfamily Orthocoronavirinae, in the family Coronaviridae, order Nidovirales, and realm Riboviria. A coronavirus is an enveloped virus with a positive-sense single-stranded RNA genome and a nucleocapsid of helical symmetry. Coronavirus refers, for example, to the following human viruses: human coronavirus 229E, human coronavirus OC43, SARS-CoV, HCoV, HKU1, MERS-CoV, and SARS-CoV-2. In some embodiments, coronavirus is SARS-CoV-2.

“Effective amount” means that amount of active compound agent that elicits the desired biological response in a subject. Such response includes alleviation of the symptoms of the disease or disorder being treated.

As used herein, the term “inflammation induced by a coronavirus infection” refers to an acute inflammation of tissues and organs that occurs as a result of a coronavirus infection. The inflammation induced by a coronavirus infection can be due to the direct viral infection of the tissues or organs, or can be due to the release of pro-inflammatory cytokines and chemokines as part of the immune reaction to the coronavirus. For example, an inflammation induced by a coronavirus infection can be acute respiratory distress syndrome (ARDS), pneumonia, myocarditis, haemophagocytic lymphohistiocytosis (sHLH), or sepsis.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

In the context of referring to the codrug according to the present invention, the term “residue” means that part of a codrug that is structurally derived from an antiviral apart from the functional group through which the moiety is linked to a compound of Formula I or Formula II. For instance, where the functional group is —NH₂, and the antiviral forms an amide (—NH—CO—) bond with a compound of Formula I or Formula I, the residue of the antiviral is that part of the antiviral that includes the —NH— of the amide, but excluding the hydrogen (H) that is lost when the amide bond is formed. Similarly, the residue of the compound of Formula I or Formula II is that part of the compounds that includes the —CO— of the amide, but excluding the hydroxyl (—OH) that is lost when the amide bond is formed. In this sense, the term “residue” as used herein is analogous to the sense of the word “residue” as used in peptide and protein chemistry to refer to a residue of an amino acid in a peptide.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition.

A “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein.

### EXAMPLES

### Example 1. Dry Powder Inhalation Pharmaceutical Composition Comprising Cromolyn and Remdesivir

Cromolyn (30 mg)+Remdesivir (50 mg) capsules for dry powder inhalation will be filled in clear #3 hydroxypropylmethylcellulose (HPMC) capsule (Qualicaps, EU S.A.), compatible with the dry powder inhaler. The ingredients of dry powder inhalation formulation are listed in Table 2.

TABLE 2
Dry Powder Inhalation of RemCromB (Remdesivir + Cromolyn) Formulation

| | | | RemCromB Composition | | | |
|---|---|---|---|---|---|---|
| | Quality | | Placebo | | Drug Product | |
| Component | Standard | Function | % w/w | mg/capsule | % w/w | mg/capsule |
| Cromolyn + Remdesivir | USP | Active | — | — | 66.6 | 80.0^{a} |
| (micronized) | | | | | | |
| Lactose monohydrate | NF | Diluent | 99.0 | 40.0 | 33.2 | 40.0 |
| Magnesium stearate | NF | Stabilizer | 1.0 | 0.4 | .03 | 0.4 |
| (micronized) | | | | | | |
| Hydroxypropyl | In-house | Encapsulation | NA | NA | NA | NA |
| methylcellulose | | | | | | |
| capsule^{b} | | | | | | |

| | | | | |
|---|---|---|---|---|
| Total | 100% | 44.4 | 100% | 120.4 |

^{a}Weight of cromolyn, USP per capsules is 30 mg + 50 mg of Remdesivir per capsule on as-is basis).
^{b}Hydroxypropyl methylcellulose capsule functions only to meter and deliver the drug product through the dry powder inhaler and is not ingested during administration.

The manufactured capsules are blistered and packaged to prevent exposure to moisture, light, and other environmental factors that could negatively impact drug stability. All product packaging and labeling will be in accordance with cGMP, GCP, local, federal, and country specific regulations and requirements.

### Example 2. Injectable Pharmaceutical Composition Comprising Remdesivir and Cromolyn

An intravenous infusion formulation comprising both remdesivir and cromolyn is produced in this example (RemCromA). A packaged white to off-white to yellow powder of Remdesivir (100 mg) and cromolyn (50 mg) mixture will be in single-dose vials, designed for reconstitution with 20 mL of sterile water for injection and diluted into 0.9% saline prior to intravenous injection. The product contains no preservative.

### Example 3. Synthesis of Chemically Bonded Antiviral Agents and Mast Cell Stabilizer (Combined AV-MCS Drug)

Briefly, an acyl chloride derivative of a molecule of group I (i.e., cromolyn) will react with the free amino group of the antiviral structure (i.e., remdesivir) to produce the amide codrug CromRemdesivir. Methods for separating the potential mono or di amide will be determined by the reactants concentrations, partial protection of the diacid or separation methods. Other methods such as DCC and modified DCC or acid anhydride activation for amidation can be used.

### Example 4. Dry Powder Inhalation Pharmaceutical Composition Comprising a Combined AV-MCS Drug

A combined AV-MCS drug obtained in Example 3 is formulated according to Example 1 to produce an inhalable dry powder inhalation formulation. The combined AV-MCS drug is the single active ingredient in the final formulation.

### Example 5. Injectable Pharmaceutical Composition Comprising a Combined AV-MCS Drug

A combined AV-MCS drug obtained in Example 3 is formulated according to Example 2 to produce an intravenous infusion formulation. The combined AV-MCS drug is the single active ingredient in the final formulation.

### Example 6. Method of Treating Animals Infected with COVID-19

Pharmaceutical compositions produced according to Examples 1-2 and 4-5 are evaluated in in vitro and in vivo studies to elucidate the mechanism of the dual action as well as, safety and efficacy of the delivery mode, such as oral, inhalation an IV infusion. Other in vitro and in vivo tests in animal models are tested to determine for viral load dampening and for decreasing proinflammatory cytokines of the codrug or the drug combination. In addition acute and chronic treatment, as well as, dose escalation in two animal species will extrapolate to dosage, safety and tolerability for human treatment..

### Example 7. Method of Treating COVID-19 Patients

A clinical trial to test for safety, tolerability, and efficacy of inhaled RemCrom is provided in this example. Dosing regimen is summarized in Table 3.

TABLE 3

| | Number of | |
|---|---|---|
| Group | subjects | Treatment |
| I | 100 | RemCrom B (q.d. inhalation of |
| | | 120.4 mg active tablet) |
| II | 100 | RemCrom B (q.d. inhalation of |
| | | two 120.4 mg active tablets) |

A minimum of 100 evaluable subjects will be randomized to receive one of two possible dosing treatment assignments various dosing of active study drug. The repeated dosing per day will be established per adverse events recorded to keep a safety profile.

Cromolyn and remdesivir are FDA-approved drugs with well-established safety profiles.

Subjects with known allergies to either of the two products will not be allowed to use the drug. Clinically significant drug interactions for co-administration of cromolyn and remdesivir, or any human drug-drug interactions with remdesivir, have not been reported.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A pharmaceutical composition comprising an antiviral agent and a compound of Formula I or Formula II: 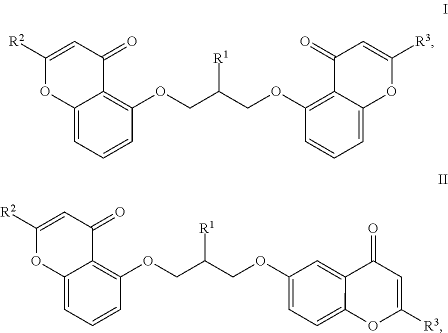
wherein
R¹ is halogen, OH, or —OC(O)C₁₋₅alkyl
R² and R³ are each independently selected from CO₂R⁴ and CH₂OR⁵;
R⁴ is Li, Na, K, H, C₁₋₅alkyl, or —CH₂CO(C₁₋₅alkyl); and
R⁵ is H or —C(O)(C₁₋₅alkyl),
or a pharmaceutically acceptable salt thereof; and
a pharmaceutically acceptable excipient.

2. The composition of claim 1, wherein the antiviral agent is selected from remdesivir (RDV), abacavir, atazanavir, bictegravir (BIC), cobicistat (GS-39250), darunavir (DRV), didanosine (ddl), dolutegravir (DTG), doravirine (MK-1439), efavirenz (EFV), elvitegravir (EVG), emtricitabine (FTC), enfuvirtide (INN), fosamprenavir, inidinavir (IDV), lamivudine (3TC), lopinavir, maraviroc, nelfinavir (NFV), Nevirapine (NVP), raltegravir (RAL), rilpivirine (TMC278), ritonavir, saquinavir (SQV), tenofovir alafenarmide (TAF), tefofovir disoproxil fumerate (TDF), tipranvir (TPV), and zidovudine (ZDV).

3. The composition of claim 1 or 2, wherein the antiviral agent is remdesivir.

4. The composition of any one of claims 1-3, wherein the compound of Formula I is cromolyn or fluorinated compound thereof or a pharmaceutically acceptable salt thereof.

5. The composition of any one of the proceeding claims, wherein the mass ratio between the antiviral agent and the compound of Formula I or Formula II is from about 10:1 to about 1:10.

6. The composition of any one of the proceeding claims, wherein the pharmaceutical composition is formulated for inhalation.

7. The composition of claim 6, wherein the composition is formulated as a dry powder for inhalation.

8. The composition of any one of claims 1 to 7, wherein the ratio between the antiviral agent and the compound is about 5:3.

9. The composition of any one of claims 1 to 5, wherein the pharmaceutical composition is formulated for intravenous injection.

10. The composition of any one of claims 1 to 5, wherein the pharmaceutical composition is formulated for intravenous infusion.

11. A codrug comprising a residue of an antiviral agent covalently bonded via a labile bond to a residue of a compound of Formula I or Formula II: 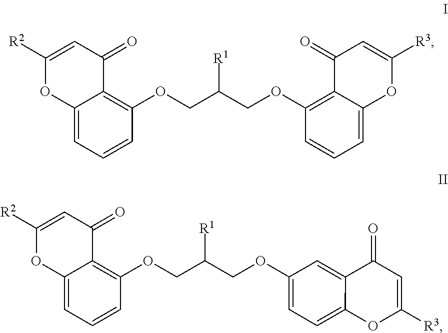
wherein
R¹ is halogen, OH, or —OC(O)C₁₋₅alkyl
R² and R³ are each independently selected from CO₂R⁴ and CH₂OR⁵;
R⁴ is Li, Na, K, H, C₁₋₅alkyl, or —CH₂CO(C₁₋₅alkyl); and
R⁵ is H or —C(O)(C₁₋₅alkyl),
or a pharmaceutically acceptable salt thereof.

12. The codrug of claim 11, wherein the residue of the antiviral agent is covalently bonded via an amide bond to a residue of a compound of Formula I or Formula II.

13. The codrug of claim 11, wherein the amide bond is formed between a functional group of the antiviral compound and a functional group at R² or R³ of the compound of Formula I or Formula II.

14. The codrug of claim 11, wherein the antiviral agent is selected from remdesivir (RDV), abacavir, atazanavir, bictegravir (BIC), cobicistat (GS-39250), darunavir (DRV), didanosine (ddl), dolutegravir (DTG), doravirine (MK-1439), efavirenz (EFV), elvitegravir (EVG), emtricitabine (FTC), enfuvirtide (INN), fosamprenavir, inidinavir (IDV), lamivudine (3TC), lopinavir, maraviroc, nelfinavir (NFV), Nevirapine (NVP), raltegravir (RAL), rilpivirine (TMC278), ritonavir, saquinavir (SQV), tenofovir alafenarmide (TAF), tefofovir disoproxil fumerate (TDF), tipranvir (TPV), and zidovudine (ZDV).

15. The codrug of claim 11, wherein the compound of Formula I or Formula II is 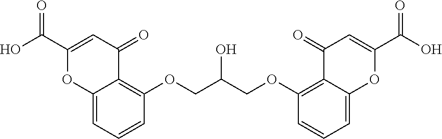 [SMILES: O=C(O)c1cc(=O)c2c(OCC(O)COc3cccc4oc(C(=O)O)cc(=O)c34)cccc2o1]
or a pharmaceutically acceptable salt thereof.

16. The codrug of claim 11, wherein the codrug is selected from remdesivir-cromolyn, abacavir-cromolyn, emtricitabine-cromolyn, lamivudine-cromolyn, tenofovir alafenamide-cromolyn, tenofovir disoproxil-cromolyn fumarate, darunavir-cromolyn, and fosamprenavir-cromolyn or a pharmaceutically acceptable salt thereof.

17. The codrug of any one of claims 11 to 16, wherein the co-drug is remdesivir-cromolyn, or fluorinated codrug thereof, or a pharmaceutically acceptable salt thereof.

18. The codrug of any one of claims 11 to 17, wherein the codrug is one of the following formulas: 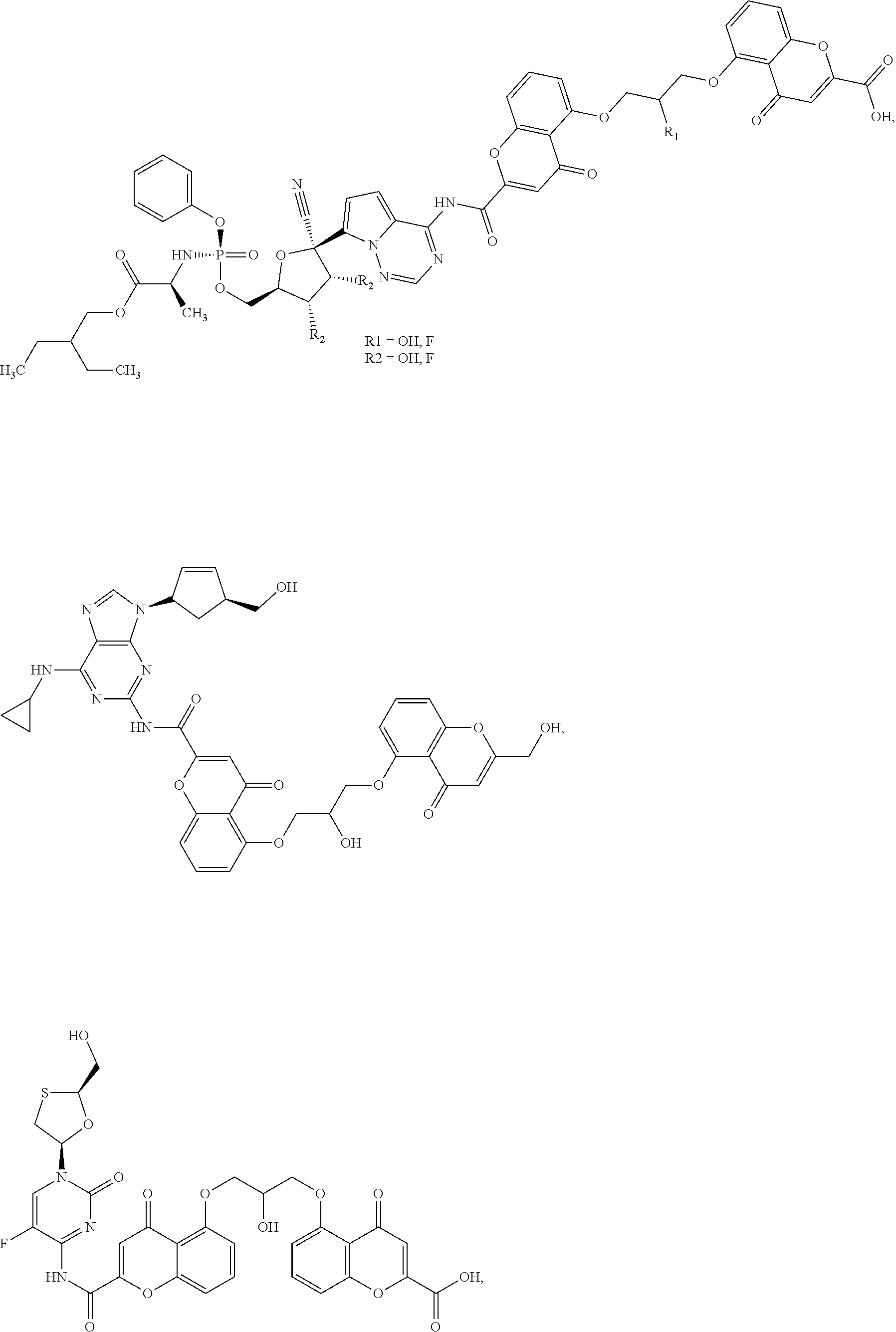 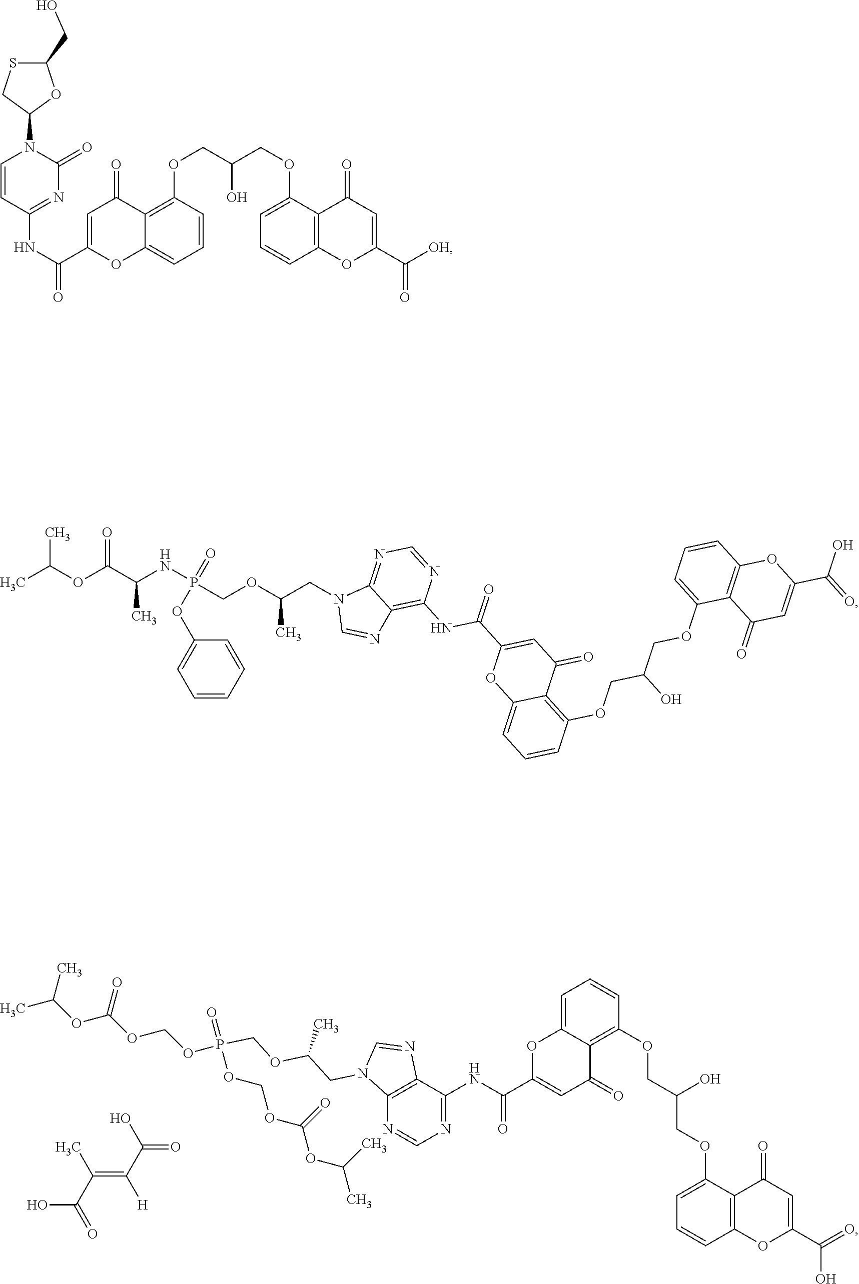 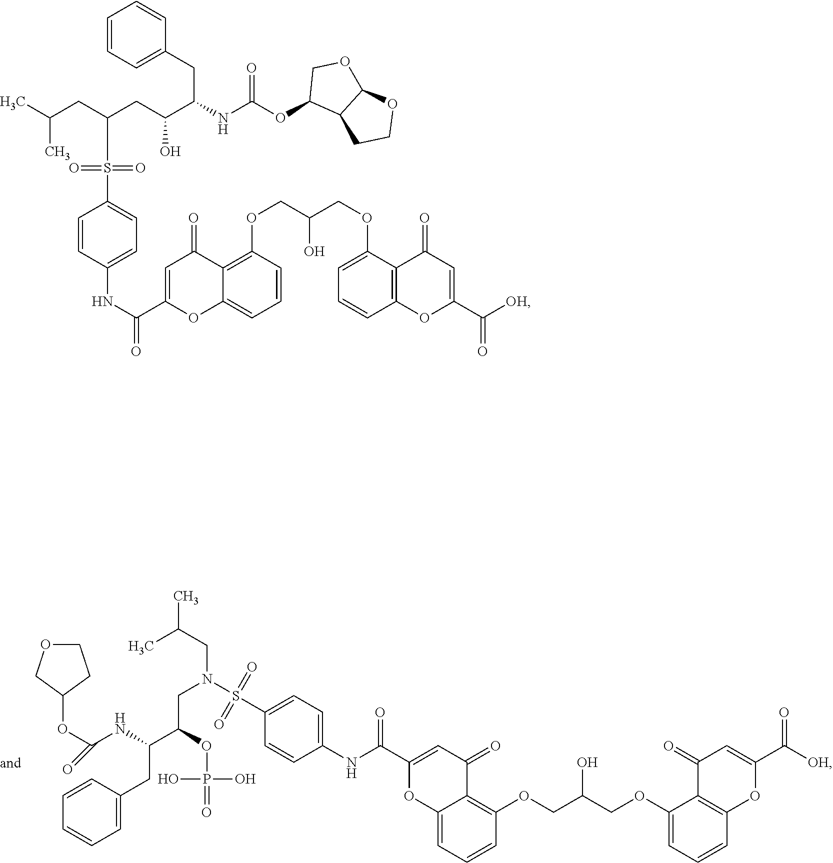 [SMILES: CC(C)CC(C[C@@H](O)[C@H](Cc1ccccc1)NC(=O)O[C@H]1CO[C@H]2OCC[C@H]21)S(=O)(=O)c1ccc(NC(=O)c2cc(=O)c3c(OCC(O)COc4cccc5oc(C(=O)O)cc(=O)c45)cccc3o2)cc1 ; CC(C)CN(C[C@@H](OP(=O)(O)O)[C@H](Cc1ccccc1)NC(=O)OC1CCOC1)S(=O)(=O)c1ccc(NC(=O)c2cc(=O)c3c(OCC(O)COc4cccc5oc(C(=O)O)cc(=O)c45)cccc3o2)cc1]
or a pharmaceutically acceptable salt thereof.

19. A pharmaceutical composition comprising a codrug of any one of claims 11 to 18 and a pharmaceutically acceptable excipient.

20. The composition of claim 19, wherein the pharmaceutical composition is formulated for inhalation.

21. The composition of claim 20, wherein the pharmaceutical composition is a dry powder for inhalation.

22. The composition of claim 19, wherein the pharmaceutical composition is formulated for injection.

23. The composition of claim 22, wherein the pharmaceutical composition is formulated for intravenous infusion.

24. The composition of claim 22 or 23, wherein the pharmaceutical composition lacks a preservative.

25. A method of treating coronavirus infection and/or associated inflammation, comprising administering an effective amount of the pharmaceutical composition of any one of claims 1 to 10, the codrug of any one of claims 11 to 18, or the pharmaceutical composition of any one of claims 19 to 24 to a subject in need thereof.

26. A method of treating coronavirus infection and/or associated inflammation, comprising administering to a subject in need thereof an effective amount of an antiviral agent and an effective amount of a compound of Formula I or Formula II: 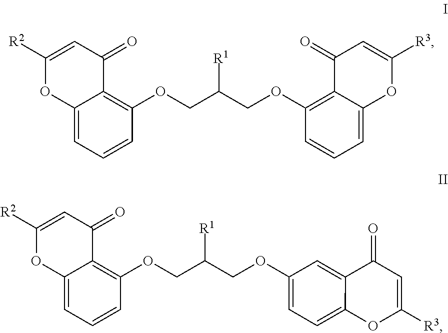
wherein
R¹ is halogen, OH, or —OC(O)C₁₋₅alkyl
R² and R³ are each independently selected from CO₂R⁴ and CH₂OR⁵;
R is Li, Na, K, H, C₁₋₅alkyl, or —CH₂CO(C₁₋₅alkyl); and
R⁵ is H or —C(O)(C₁₋₅alkyl),
or a pharmaceutically acceptable salt thereof.

27. The method of claim 26, wherein the antiviral agent is selected from remdesivir (RDV), abacavir, atazanavir, bictegravir (BIC), cobicistat (GS-39250), darunavir (DRV), didanosine (ddl), dolutegravir (DTG), doravirine (MK-1439), efavirenz (EFV), elvitegravir (EVG), emtricitabine (FTC), enfuvirtide (INN), fosamprenavir, inidinavir (IDV), lamivudine (3TC), lopinavir, maraviroc, nelfinavir (NFV), Nevirapine (NVP), raltegravir (RAL), rilpivirine (TMC278), ritonavir, saquinavir (SQV), tenofovir alafenarmide (TAF), tefofovir disoproxil fumerate (TDF), tipranvir (TPV), and zidovudine (ZDV).

28. The method of claim 26 or 27, wherein the antiviral agent is remdesivir.

29. The method of any one of claims 26 to 28, wherein the compound of Formula I is cromolyn or a pharmaceutically acceptable salt thereof.

30. The method of any one of claims 26 to 29, wherein the mass ratio between the antiviral agent and the compound of Formula I or Formula II is from about 10:1 to about 1:10.

31. The method of any one of claims 26 to 30, wherein the antiviral agent and the compound of Formula I or Formula II are administered by intravenous infusion, by intravenous injection, by subcutaneous injection, by intramuscular injection, or by intraperitoneal injection.

32. The method of any one of claims 26 to 31, wherein the antiviral agent and the compound of Formula I or Formula II are administered by inhalation.

33. The method of any one of claims 26-32, wherein the antiviral agent and the compound of Formula I or Formula II are administered by dry powder inhalation.

34. The method of claim 33, wherein the mass ratio between the antiviral agent and the compound of Formula I or Formula II is about 5:3.

35. The method of any one of claims 26-30, wherein the antiviral agent and the compound of Formula I or Formula II are administered by intravenous infusion or by intravenous injection.

36. The method of claim 25, wherein the mass ratio between the antiviral agent and the compound of Formula I or Formula II is about 2:1.

37. The method of any one of claims 25 to 36, wherein the coronavirus is selected from SARS-CoV, MERS-CoV, HCoV, HKU1, and SARS-CoV-2.

38. The method of any one of claims 25 to 36, wherein the coronavirus is selected from SARS-CoV, MERS-CoV, and SARS-CoV-2.

39. The method of any one of claims 25 to 36, wherein the coronavirus is SARS-CoV-2.

40. The method of any one of claims 25 to 39, wherein the one or more inflammation are selected from acute respiratory distress syndrome (ARDS), pneumonia, myocarditis, haemophagocytic lymphohistiocytosis (sHLH), kidney failure, septic shock, and sepsis.

41. The method of claim 40, wherein at least one inflammation condition is pneumonia.

42. The method of claim 40, wherein at least one inflammation condition is ARDS.

43. The method of claim 40, wherein at least one inflammation condition is myocarditis.

44. The method of claim 40, wherein at least one inflammation condition is sepsis.

45. The method of any one of claims 25 to 44, wherein the subject is aged 18-75 years, inclusive.

46. The method of any one of claims 25 to 45, wherein the subject has SARS-CoV-2 infection, which has been confirmed by reverse-transcription polymerase chain reaction (RT-PCR) from respiratory tract or blood specimens.
